(19) United States
(12) Patent Application Publication
(10) Pub. No.: US 2008/0274470 A1
(43) Pub. Date: Nov. 6, 2008

(54) NON-INVASIVE DETECTION OF ENDOMETRIAL CANCER

(75) Inventors: Quynh Hoa Le, Woodridge, IL (US); Herbert R. Radisch, San Diego, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: BIOCEPT, INC., San Diego, CA (US)

(21) Appl. No.: 12/114,584

(22) Filed: May 2, 2008

Related U.S. Application Data
(60) Provisional application No. 60/915,554, filed on May 2, 2007.

(51) Int. Cl. C12Q 1/68; C12Q 1/04; G01N 33/574; C12Q 1/06; C12N 5/06

(52) U.S. Cl. 435/6; 435/34; 435/7.23; 435/39; 435/325

(57) ABSTRACT

The present invention provides a non-invasive method of obtaining a sample of endometrial cells for use in the diagnosis of endometrial cancer, as well as methods and kits for diagnosing, determining the prognosis of, and monitoring endometrial cancer.

The present application claims priority to U.S. Provisional Application No. 60/915,554, filed May 2, 2007, which is incorporated by reference herein in its entirety.

### FIELD OF THE INVENTION

This invention relates generally to detection and monitoring of endometrial growth, especially neoplastic growth of endometrial cells.

### BACKGROUND OF THE INVENTION

Endometrial cancer involves, among other things, neoplastic growth of the endometrium, the inner lining of the uterus. It is the most common gynecologic cancer in the United States, with over 35,000 women being diagnosed each year.

Endometrial biopsy is currently the most widely used screening technique for the diagnosis of endometrial cancer. This procedure involves inserting a narrow tube into the uterus through the vagina and suctioning out a small amount of tissue from several areas of the uterine wall. The tissue is examined under a microscope and evaluated for cancerous or pre-cancerous abnormalities. This procedure provides an accurate diagnosis in 90% of cases. A transvaginal ultrasound may be done before the biopsy to help locate particular areas that should be biopsied.

In some cases, for example, if the endometrial biopsy was not conclusive, a hysteroscopy can be performed. This is a technique in which a tiny telescope, that allows doctors to look inside the uterus, is inserted into the uterus through the cervix . The uterus is then expanded by filling it with saline, thereby enabling doctors to see what might be causing any bleeding, such as a cancer or a polyp. The abnormality can then be biopsied. This procedure is typically performed with local anesthesia.

Alternatively, a dilatation and curettage (D & C) can be performed to diagnose the disease. This procedure involves dilating the cervix and inserting an instrument called a curette into the uterus through the vagina. The curette is used to scrape the uterine wall and collect tissue. In suction curettage, suction is applied through a narrow tube to remove the tissue sample. Although D & C is typically performed as an outpatient procedure, it requires general anesthesia.

In addition, imaging tests can be performed in conjunction with, or in addition to, the above procedures in order to better identify the region to be biopsied, to check for the extent of spread of the cancer, or where the patient may be unable to safely tolerate the general anesthesia. Commonly performed imaging tests include transvaginal ultrasound or sonography, transabdominal ultrasound, Computed Tomography (CT) scan, and Magnetic Resonance Imaging (MRI) scan and Positron Emission Tomography (PET) scan.

There is still a need in the field to provide additional means to obtain endometrial samples and to monitor or detect endometrial growth.

### SUMMARY OF THE INVENTION

The present invention is based, at least in part, on the discovery that menstrual samples can be used for detection and monitoring of endometrial cells. Accordingly, the present invention provides methods and kits useful for screening endometrial cancer or monitoring endometrial cell growth.

In one embodiment, the present invention provides methods of screening for endometrial cancer in a subject. The method comprises examining a menstrual sample obtained from the subject and determining the presence or absence of a neoplastic cell in the menstrual sample, wherein the presence of a neoplastic cell in the menstrual sample is indicative of endometrial cancer. In certain embodiments, the number of neoplastic cells or the neoplastic cell markers present in the menstrual sample provides a prognosis relating to endometrial cancer in the subject.

In another embodiment, the present invention provides methods for monitoring the development of endometrial cancer in a subject. The method comprises examining a menstrual sample obtained from the subject and determining the presence or absence of neoplastic cells in the menstrual sample.

In yet another embodiment, the present invention provides methods for obtaining an endometrial sample of a subject. The method comprises obtaining a menstrual sample of the subject, wherein the menstrual sample of the subject is an endometrial sample of the subject.

In still another embodiment, the present invention provides kits useful for examining an endometrial sample of a subject. The kit comprises a menstrual sample of the subject and, optionally, an instruction, e.g., for using the menstrual sample as a source endometrial sample or for detecting neoplastic endometrial cells or markers associate with neoplastic endometrial cells.

### DETAILED DESCRIPTION OF THE INVENTION

The present invention is based, at least in part, on the discovery that menstrual samples can be used for detecting and monitoring endometrial cells. In particular, during menstruation the endometrium, containing substantially the entire endometrial cell compliment, is shed as a liquid called the menses that flows through the cervix and vagina. Thus, the menses can be used as a source of endometrial cells and, e.g., collected non-invasively for examining endometrial cells. Accordingly, the present invention provides methods and kits useful for screening endometrial cancer or monitoring endometrial cell growth.

According to one aspect of the present invention, methods for obtaining an endometrial sample of a subject, e.g., a human subject, by obtaining a menstrual sample of the subject are provided. The term “menstrual sample,” used interchangeably herein with “menses” or “menstrual flow,” and also known as catamenia or a period, refers to the monthly flow of blood and cellular debris from the uterus that begins at puberty in human women and the females of other primates, and ceases at menopause in human women.

According to the present invention, a menstrual sample can be any biological sample obtained from the menstrual flow of a subject. For example, the menstrual sample of the present invention can be an isolated cell sample including cells isolated from the menstrual flow of a subject. Alternatively, the menstrual sample of the present invention can be an isolated nucleic acid or protein sample obtained from the menstrual flow of a subject. In one embodiment, the menstrual sample of the present invention is an isolated cell sample suitable for imaging assays, e.g., flow cytometry analysis. In another embodiment, the menstrual sample of the present invention is an isolated nucleic acid sample suitable for nucleic acid screening assays, e.g., PCR, hybridization, etc. In yet another embodiment, the menstrual sample of the present invention is an isolated protein sample suitable for protein binding assays, e.g. Western, gel shift, ELISA, etc.

The menstrual sample can be obtained from any source and by any suitable means known or later discovered in the field. In general, most women use some means or devices to absorb or collect their menses to prevent soiling their clothes. Thus, in one embodiment, the menstrual sample for use in the invention is obtained from any means or device used for sanitary reasons. Means or devices that absorb the menstrual flow may either be disposable or re-usable. Examples of disposable means that absorb the menstrual flow include, but are not limited to, sanitary napkins (also called sanitary towels, sanitary pads, maxi pads or pads), liners, tampons and padettes. Examples of re-usable means that absorb the menstrual flow include, but are not limited to, re-usable cloth pads (e.g., lunapads), sea sponges, padded panties, and large re-useable cloth. Depending on whether the absorbent means are disposable or re-usable, they may be made of absorbent materials such as wood pulp, wood cellulose fibers, hemp, treated rayon/cotton blend, fleece, cotton, terrycloth, or flannel.

In another embodiment of the invention, menstrual samples useful for the present invention can be obtained from any means or device specially designed for collecting menstrual samples. Examples of such means include, but are not limited to, menstrual cups. Menstrual cups may either be disposable (e.g., Instead® or Instead® Softcup®) or re-usable (e.g., The Keeper®, The Mooncup® DivaCup™, Lunette™, etc.) and are made of plastic (disposable), silicone or latex (re-usable).

Biological samples can be isolated from these sanitary materials or collection devices via any means known or later discovered in the art. For example, menstrual cell samples can be obtained by eluting and/or isolating cells from the absorbent material containing the menstrual sample. The absorbent material can be soaked in a liquid, for example, water or saline, to separate the menses from the absorbent material. Alternatively, pressure can be applied to the absorbent material so as to squeeze it, and thereby separate the menses from the absorbent material. Any sample containing cells derived from the menses can be further purified through various methods, e.g., binding assays, size selection, etc. Such a sample can also be further processed to obtain a particular component of the sample, e.g., nucleic acid, protein, membrane, mitochondria, nuclei, cytoplasm, etc.

In one embodiment, nucleic acid material is isolated from the menstrual sample. A variety of techniques for isolating nucleic acids from biological samples are known in the art. For example, see the techniques described in Sambrook et al., 1989, Molecular Cloning: A Laboratory Manual, Cold Spring Harbor Laboratory, 1989, 2nd ed., NY; id., 3rd ed., 2001. The nucleic acid material can be DNA, RNA, or a combination thereof. The quantity and concentration of nucleic acid required can vary, and will be apparent to those of skill in the art.

In certain embodiments, the nucleic acid obtained can be amplified by methods such as polymerase chain reaction (“PCR”) or other known or later developed amplification techniques. PCR enables one to amplify a specific region of a nucleic acid of interest, e.g., from a mixture of nucleic acids, when one or more copies of the nucleic acid of interest is present in a sample. PCR primers can contain restriction enzyme recognition sequences so that amplified nucleic acids can be manipulated by the corresponding restriction enzyme. Amplification of a nucleic acid of interest can occur prior to or concurrent with detection or assessment of a nucleic acid sequence associated with endometrial neoplastic cells.

According to another aspect of the present invention, methods for screening for endometrial cancer in a subject, by examining a menstrual sample obtained from the subject and determining the presence or absence of a neoplastic cell in the menstrual sample, are provided.

The presence of neoplastic cells can be detected by any presently known or later discovered method for detecting cells with neoplastic characteristics. According to the present invention, a neoplastic cell means any cell with abnormal growth, e.g., cells with out of control proliferation including, without any limitation, abnormal proliferating cells that remain clustered together, e.g., benign tumors, or abnormal proliferating cells capable of breaking lose and entering the bloodstream or lymphatic vessels to form secondary tumors or metastases at other sites in a subject, e.g., malignant tumors.

In general, the presence of neoplastic cells can be detected by various cytogenetic, nucleic acid, protein, or immunochemical analyses. For example, the presence of neoplastic cells can be detected by direct examination of the morphology of cells in a menstrual sample or by contacting a menstrual sample with a probe that detects neoplastic endometrial cells or assesses one or more markers that are qualitatively or quantitatively associated with neoplastic endometrial cells. For example, the presence of neoplastic endometrial cells can be detected by contacting a menstrual sample with a ligand capable of specifically binding to or interacting with an entity, the presence or absence of which or an increase or decrease in its level of presence is specifically associated with neoplastic endometrial cells.

In one embodiment, the ligand in contact with a menstrual sample is an antibody, which recognizes an epitope specifically associated with neoplastic endometrial cells, e.g., cell surface markers for neoplastic endometrial cells. The antibody can be an antibody binding region, CDR, single chain antibody, chimeric antibody, or humanized antibody. The antibody can also be a monoclonal antibody or polyclonal antibody. Examples of antibodies useful for identifying neoplastic endometrial cells include, but are not limited to, MSN-1 antibodies, OXA antibodies, OXB antibodies, PTEN antibodies, anti-LeY antibodies, anti-CAGE antibodies, anti-UPAR antibodies, anti-Hepcidin antibodies, and anti-KLK4 antibodies.

Any method or technique suitable for immuno-detection can be used in association with the present invention. For example, such methods include using competitive and non-competitive assay systems with techniques such as radio-immunoassay (RIA), enzyme linked immunosorbent assay (ELISA), sandwich immunoassay, immunoradiometric assay, gel diffusion precipitation reaction, immunodiffusion assay, in situ immunoassay (using colloidal gold, enzyme or radioisotope labels, for example), Western blot, precipitation reactions, agglutination assays (e.g., gel agglutination assays), complement fixation assay, immunofluorescence assay and immunoelectrophoresis assay.

In another embodiment, the ligand in contact with a menstrual sample is a peptide or polypeptide (protein) capable of specifically interacting with a peptide or polypeptide specifically associated with neoplastic endometrial cells, e.g., peptide and/or protein markers for neoplastic endometrial cells. For example, a peptide and/or protein marker for neoplastic endometrial cells can be one or more peptides, peptide regions, or proteins, e.g., in their native or modified forms specifically associated with one or more mutations or genes expressed in neoplastic endometrial cells. Alternatively, a peptide and/or protein marker for neoplastic endometrial cells can be one or more peptides, peptide regions, or proteins in a particular modified form or state, e.g., forms or states associated with activation, phosphorylation, association with another entity, glycosylation, translocation, etc. that is specifically associated with endometrial neoplastic cells. Examples of peptide and/or protein markers for neoplastic endometrial cells include, but are not limited to, SSX protein family members, e.g., SSX1, SSX4 and SSX5 or fragments thereof, MSN-1, OXA, OXB, PTEN, LeY, CAGE, UPAR, Hepcidin, and KLK4.

In yet another embodiment, the ligand in contact with a menstrual sample is a nucleic acid probe capable of specifically hybridizing with a nucleic acid sequence specifically associated with neoplastic endometrial cells, e.g., molecular and/or nucleic acid markers for neoplastic endometrial cells. For example, a molecular and/or nucleic acid marker for neoplastic endometrial cells can be one or more SNPs, mutations, intron-exon splicing arrangements, transcripts of genes associated with neoplastic endometrial cells, genetic sequences or genes associated with neoplastic endometrial cells, etc.

According to the present invention, the term “hybridization” or “hybridizing” refers to any process by which a nucleic acid, e.g., probe, binds, partially or entirely, to another nucleic acid in a sequence complementary manner through nucleic acid base pairing. Hybridization conditions can be defined by, for example, the concentrations of salt or formamide in the prehybridization and hybridization solutions, or by the hybridization temperature, and are well known in the art. Hybridization can occur under conditions of various stringency, e.g., high, medium, or low stringency conditions. Conditions required to ensure stringent hybridization are well known in the art, and are described, for example, in Sambrook et al., supra and Ausubel et al., 1994, Current Protocols in Molecular Biology, Greene Publishing Associates and Wiley Interscience, NY. Typically, stringent conditions for short probes (e.g., 10 to 50 nucleotides) will be those in which the salt concentration is at least about 0.01 to 1.0 M at pH 7.0 to 8.3 and the temperature is at least about 30° C. Stringent conditions can also be achieved with the addition of destabilizing agents such as formamide. The hybridization can then be detected by methods known to one of skill in the art.

Any method or technique suitable for detecting a nucleic acid sequence, qualitatively or quantitatively, can be used in association with the present invention. Such methods include, but are not limited to, restriction-fragment-length-polymorphism detection based on gene-specific restriction-endonuclease cleavage; mismatch-repair detection; binding of MutS protein; denaturing-gradient gel electrophoresis; single-strand-conformation-polymorphism detection; RNAase cleavage at mismatched base-pairs; chemical or enzymatic cleavage of heteroduplex DNA; methods based on gene-specific primer extension; genetic bit analysis; oligonucleotide-ligation assay; oligonucleotide-specific ligation chain reaction (“LCR”); gap-LCR; radioactive or fluorescent DNA sequencing using standard procedures well known in the art; and peptide nucleic acid (PNA) assays.

Other methods of detecting a nucleic acid sequence associated with endometrial neoplastic cells include detecting an amplicon from an amplification reaction of a menstrual sample. Examples of amplification reactions include, but are not limited to, polymerase chain reaction (“PCR”), real-time polymerase chain reaction (“RT-PCR”), ligase chain reaction (“LCR”), self-sustained sequence replication (“3SR”) also known as nucleic acid sequence based amplification (“NASBA”), Q-B-Replicase amplification, rolling circle amplification (“RCA”), transcription mediated amplification (“TMA”), linker-aided DNA amplification (“LADA”), multiple displacement amplification (“MDA”), invader and strand displacement amplification (“SDA”). In addition, amplification reactions can be carried out using labeled or modified nucleotides which are detectable either directly or via another reaction. For example, RNAs can be amplified using labeled UTP, e.g., aminoallyl-UTP as well as free UTP and the aminoallyl groups incorporated into the amplified RNA can be reacted with a fluorescent chromophore, such as CyDye for detection.

According to the present invention, the term “a nucleic acid sequence”, “a nucleic acid” or “nucleic acid probe” refers to any nucleic acid comprising, for example, from about 5 nucleotides to about 10, 20, 30, 40, 50, 60, 70, 80, 90, 100, 150, 200, 250, 300, 400, 500, 600, 700, 800, 900, 1000, or more nucleotides. The terms encompass naturally occurring, synthetic, and modified nucleotides. The terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms include nucleic acids that have been modified in order to introduce a means for attachment (e.g., for use in a microarray).

In some embodiments, a nucleic acid or probe can be immobilized on a support, for example, in an array or a microarray. “Array” or “microarray,” as used herein, comprises a surface with an array, preferably an ordered array, of putative binding (e.g., by hybridization) sites for a biochemical sample which often has undetermined characteristics. An array can provide a medium for matching known and unknown nucleic acid molecules based on base-pairing rules and automating the process of identifying the unknowns. An array experiment can make use of common assay systems such as microplates or standard blotting membranes, and can be worked manually, or make use of robotics to deposit the sample. The array can be a macro-array (containing nucleic acid spots of about 250 microns or larger) or a micro-array (typically containing nucleic acid spots of less than about 250 microns).

Still other methods of detecting a nucleic acid sequence associated with endometrial neoplastic cells include sequencing the nucleic acid obtained from the menstrual sample of a subject. For example, the DNA obtained from a subject can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. Other available techniques are described in Sambrook et al., supra, and Ausubel et al., supra.

According to the present invention, the ligand in contact with a menstrual sample for detection of neoplastic endometrial cells can be labeled with any reagent suitable for detection purposes. Such a labeling reagent can be any known or later discovered detectable entity including, but not limited to, various enzymes, prosthetic groups, fluorescent labels, chemiluminescent labels, bioluminescent labels, and radioactive labels. Examples of suitable enzymes include, but are not limited to, horseradish peroxidase, alkaline phosphatase, β-galactosidase, α-glycerophosphate, aspariginase, glucose-6-phosphate dehydrogenase, glucoamylase, glucose oxidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride, allophycocyanin, green fluorescent protein, o-phthaldehyde, phycocyanin and phycoerythrin. Examples of chemiluminescent materials include, but are not limited to, acridinium salt, imidazole, oxalate ester, theromatic acridinum ester, luminol and isoluminol. Examples of bioluminescent materials include, but are not limited to, luciferase, luciferin, and aequorin. Examples of suitable radioactive material include, but are not limited to, 125I, 131I, 35S, 32P and 3H.

The ligand can be immobilized to a solid surface or in suspension. In one embodiment, the ligand is immobilized on the surface of an apparatus so that it can bind endometrial neoplastic cells via their cell surface marker(s). In another embodiment, the ligand is immobilized via a linker to the surface of an apparatus suitable for the ligand's interaction with any marker associated with endometrial neoplastic cells. In yet another embodiment, the ligand is coated on the surface of an apparatus.

In one embodiment, the apparatus is a microflow device which comprises an inlet means, an outlet means, and a microchannel arrangement extending between the inlet and outlet means. The microchannel arrangement can be any microchannel capable of providing a randomized flow path for the menstrual sample. In one embodiment, the microchannel arrangement includes a plurality of transverse separator posts that are integral with a base surface of the microchannel and project therefrom. The posts are generally arranged in a pattern capable of providing a randomized flow path. Examples of microflow devices are described in U.S. application Ser. Nos. 11/458,668 and 11/331,988, both of which are incorporated herein in their entirety.

The surface of the microchannel arrangement of the microflow device can be coated, partially or entirely, with at least one ligand capable of specifically binding to, or interacting with, an entity specifically associated with neoplastic endometrial cells. The ligand can be a nucleic acid, a polypeptide or an antibody or a fragment thereof, as discussed above. In one embodiment, the ligand is an antibody or a fragment thereof.

According to yet another aspect of the present invention, it provides methods for monitoring the development of endometrial cancer in a subject by examining a menstrual sample obtained from the subject and determining the presence or absence of neoplastic cells in the menstrual sample. For example, the effectiveness of a treatment for endometrial cancer can be determined by monitoring the development of endometrial cancer in a subject under the treatment, e.g., by examining one or more menstrual samples obtained from the subject prior to, during, and/or after the treatment and determining the presence or absence of neoplastic cells in the menstrual sample, including without any limitation, determining the presence or level, e.g., quantitative level, of neoplastic cells in the menstrual sample.

According to still another aspect of the present invention, it provides kits useful for examining the endometrial sample of a subject comprising one or more menstrual samples of the subject and optionally instruction(s), e.g., for using the menstrual sample as a source of endometrial sample or for detecting neoplastic endometrial cells or markers associated with neoplastic endometrial cells. As used herein, the term “kit” refers to a container or device, optionally including an instruction and/or one or more reagents, e.g., one or more antibodies, nucleic acid probes, primers, ligands, peptides, etc., to be used in connection with the menstrual sample contained therein.

Although the invention has been described with reference to the presently preferred embodiments, it should be understood that various modifications can be made without departing from the spirit of the invention. Accordingly, the invention is limited only by the following claims.

What is claimed is:

1. A method of screening for endometrial cancer in a subject comprising
examining a menstrual sample obtained from the subject and
determining the presence or absence of a neoplastic cell in the menstrual sample,
wherein the presence of a neoplastic cell in the menstrual sample is indicative of endometrial cancer.

2. The method of claim 1, wherein the menstrual sample is a cell sample isolated from the menses of the subject.

3. The method of claim 1, wherein the menstrual sample is a nucleic acid or protein sample isolated from the menses of the subject.

4. The method of claim 1, wherein the presence or absence of a neoplastic cell in the menstrual sample is determined by detecting the presence or absence of a nucleic acid sequence associated with neoplastic growth of endometrial cells.

5. The method of claim 1, wherein the presence or absence of a neoplastic cell in the menstrual sample is determined by detecting the presence or absence of an amino acid sequence associated with neoplastic growth of endometrial cells.

6. The method of claim 1, wherein the presence or absence of a neoplastic cell in the menstrual sample is determined by detecting the presence or absence of an epitope associated with neoplastic growth of endometrial cells.

7. The method of claim 1, wherein the presence or absence of a neoplastic cell in the menstrual sample is determined by using an antibody that specifically binds to an epitope associated with neoplastic growth of endometrial cells.

8. The method of claim 7, wherein the antibody is immobilized on a solid support.

9. The method of claim 7, wherein the antibody is coated on the surface of a microchannel arrangement of a microflow device,
wherein the microflow device comprises a body having a randomized flow path which comprises an inlet means, an outlet means, and the microchannel arrangement extending between said inlet and outlet means,
wherein the microchannel arrangement includes a plurality of transverse separator posts being integral with a base surface of said microchannel and projecting therefrom, and
wherein said posts are arranged in a pattern capable of providing the randomized flow path.

10. A method for monitoring the development of endometrial cancer in a subject comprising examining a menstrual sample obtained from the subject and determining the presence or absence of neoplastic cells in the menstrual sample.

11. The method of claim 10, wherein the presence of neoplastic cells is determined quantitatively.

12. The method of claim 10, wherein the menstrual sample is a cell sample isolated from the menses of the subject.

13. The method of claim 10, wherein the menstrual sample is a nucleic acid or protein sample isolated from the menses of the subject.

14. The method of claim 10, wherein the presence or absence of neoplastic cells in the menstrual sample is determined by detecting the presence or absence of a nucleic acid sequence associated with neoplastic growth of endometrial cells.

15. The method of claim 10, wherein the presence or absence of a neoplastic cell in the menstrual sample is determined by detecting the presence or absence of an amino acid sequence associated with neoplastic growth of endometrial cells.

16. The method of claim 10, wherein the presence or absence of a neoplastic cell in the menstrual sample is determined by detecting the presence or absence of an epitope associated with neoplastic growth of endometrial cells.

17. A method for obtaining an endometrial sample of a subject comprising obtaining a menstrual sample of the subject, wherein the menstrual sample of the subject is an endometrial sample of the subject.

18. A kit useful for examining an endometrial sample of a subject comprising a menstrual sample of the subject and an instruction.

19. The kit of claim 18, wherein the menstrual sample is a cell sample isolated from the menses of the subject.

20. The kit of claim 18, wherein the menstrual sample is a nucleic acid or protein sample isolated from the menses of the subject.

21. The kit of claim 18, further comprising a pair of oligonucleotides suitable for amplifying a nucleic acid sequence associated with neoplastic growth of endometrial cells.

22. The kit of claim 18, further comprising an antibody capable of specifically binding to an epitope associated with neoplastic growth of endometrial cells.

23. The kit of claim 18, further comprising a ligand capable of specifically binding to an amino acid sequence associated with neoplastic growth of endometrial cells.
